(19) United States
(12) Patent Application Publication
(10) Pub. No.: US 2025/0050124 A1
(43) Pub. Date: Feb. 13, 2025

(54) SYSTEMS FOR AUTOMATED OR MANUAL TRANSCRANIAL MAGNETIC STIMULATION

(71) Applicant: United States Government as Represented by the Department of Veterans Affairs, Washington, DC (US)

(72) Inventor: Punit Vaidya, Cleveland, OH (US)

(21) Appl. No.: 18/797,179

(22) Filed: Aug. 7, 2024

Related U.S. Application Data
(60) Provisional application No. 63/518,019, filed on Aug. 7, 2023.

(51) Int. Cl. A61N 2/00; A61N 2/02

(52) U.S. Cl. CPC A61N 2/006; A61N 2/02

(57) ABSTRACT

A system includes a magnetic stimulation coil; and a machine vision navigation device comprising a camera configured to capture successive still images and/or to stream video indicating position of the magnetic stimulation coil. The machine vision navigation device is configured to determine, based on the successive still images and/or the video, the position of the magnetic stimulation coil relative to a target treatment area of a patient, and cause, based on the position of the magnetic stimulation coil relative to the target treatment area of the patient, display of an indication of the position of the magnetic stimulation coil relative to the target treatment area of the patient.

### CROSS-REFERENCE TO RELATED APPLICATIONS

This application claims priority benefit of U.S. Provisional Application No. 63/518,019, filed Aug. 7, 2023, the entirety of which is incorporated herein by reference.

### BACKGROUND

Transcranial magnetic stimulation is a non-invasive method of brain stimulation that utilizes high-intensity magnetic pulses to activate neurons. When pulses are administered repetitively (referred to as repetitive transcranial magnetic stimulation, or rTMS), it can induce long-lasting changes in neuronal excitability to produce therapeutic results. rTMS may be used for the treatment of medication-resistant major depressive disorder, but it is also approved for other indications, including obsessive compulsive disorder and smoking cessation. It is being investigated for many other conditions including posttraumatic stress disorder, stroke rehabilitation, pain conditions, cognitive disorders, and others. rTMS may be very safe and well-tolerated, and unlike medications, may eliminated one or more systemic side-effects, such as weight gain, gastrointestinal side-effects, or sexual side dysfunction. Patients may be awake and alert for the treatment and can resume their usual activities after each session, because rTMS may not require anesthesia or sedation or carry the risk of cognitive side effects. This allows patients to drive to and from the treatment location, which is typically a clinic or hospital setting.

However, rTMS protocols are time-intensive. For depression, a standard protocol may include treatments administered 5 days a week for 4-6 weeks, with an additional 3-week taper in responders. Despite the excellent safety and tolerability of rTMS, the demanding treatment schedule can be daunting for many patients. For patients who have caregiving responsibilities, live a far distance from the TMS clinic, are employed full-time, or have physical impairments, the travel and scheduling requirements may be prohibitive. Additionally, those who are depressed may lack the energy or motivation to follow through with such a demanding treatment protocol. Thus, at-home TMS systems are needed, as they may increase the accessibility of rTMS therapy.

### SUMMARY

Described herein, in various aspects, are methods, an apparatus, and systems configured for repetitive transcranial magnetic stimulation (rTMS) in an automated or manual fashion and in various settings, including in the clinic or at-home. rTMS may be a treatment option for conditions such as major depressive disorder and other mental health conditions. The system includes a portable navigation device that enables consistent manual or automated positioning and orientation of the TMS coil on the target of the patient. Along with ensuring precise and optimal coil positioning, the system may include multiple safety features, a method for remote delivery and monitoring of the treatment, and a mechanism for assessing clinical response.

Additional advantages of the invention will be set forth in part in the description that follows, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a depiction of an example TMS system in accordance with embodiments described herein.

FIG. 2 is a depiction of an example controller permitting patient control of a TMS system and for safety monitoring in accordance with embodiments described herein.

FIG. 3 is a depiction of an example wrist controller permitting patient control of a TMS system and for safety monitoring in accordance with embodiments described herein.

FIG. 4 is a depiction of an example machine vision navigation device retrofitted onto a previously constructed prototype TMS coil with an alignment device in accordance with embodiments described herein.

FIG. 5 is a depiction of the example machine vision navigation device of FIG. 4 of the alignment markings on the base plate and the coupled TMS coil in accordance with embodiments described herein.

FIGS. 6A-6C depict various view of an example implementation of a cap in accordance with embodiments described herein.

FIGS. 7A-7F depict various images of a camera feeds after processing by a machine vision algorithm in accordance with embodiments described herein.

FIG. 7G depicts an image of the image of FIG. 7E from a different view, in accordance with embodiments described herein.

FIG. 8 depicts an example embodiment of a TMS coil in accordance with embodiments described herein.

FIGS. 9A and 9B depict a various views of an example embodiment of a TMS coil in accordance with embodiments described herein.

FIG. 10 depicts a system including a computing device for use with the system of FIG. 1 in accordance with embodiments described herein.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, flash drive, SD card or similar non-volatile memory card, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a microcontroller, general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a microcontroller, computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems or one or more microcontrollers that perform the specified functions or steps, or combinations of special purpose hardware and computer instructions.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

Interest in at-home TMS has recently emerged in the field, especially since the start of the COVID-19 pandemic. There are some challenges with implementing at-home TMS, primarily as it relates to the ability for the system to maintain proper position and orientation of the coil with respect to the target, assessing quality of coil contact throughout treatment, assessing clinical response, and more importantly, ensuring safe delivery of the therapy.

Typically, clinical rTMS is done in a hospital or office-based setting, under the direct observation of a TMS clinician, typically a physician, nurse, or TMS technician. The TMS operator may ensure the patient is properly seated, that safety measures are followed (such as removal of metal jewelry, donning of earplugs) and positions the coil over the desired target. The operator sets the appropriate stimulation parameters, initiates the stimulation, pauses the treatment if needed, and continuously monitors the patient for any adverse events. Of particular importance is that the clinician must recognize and respond to a seizure. Some systems include neuronavigation, which relies on a complex imaging and computational system with multiple cameras and optical trackers which are attached to the coil and the patient's head. The system creates a 3-dimensional reconstruction of the coil location and orientation over an MRI image of the patient's brain or a generic head model. Such a technique may be costly, time-intensive, and not practical for routine clinical application. Thus, in clinical practice, visual approximation may be used to place the MS coil and is the technique employed in most of the devices that have been FDA approved for the treatment of major depression. However, this method may be crude and imprecise, because it may not provide objective confirmation of proper coil placement or contact with the target. This method may also be prone to errors, and can compromise clinical efficacy. Many of the methods for MS coil placement rely on a cap, resembling a swimmer's cap, which is fit snuggly over the patient's head. The target is marked on the cap, and the coil is centered over the target at the start of each treatment. However, due to the dimensions of the coil, once placed on the head, the target marking and center of the underside of the coil are occluded from visual inspection, so the location of the edge of the coil is marked on the cap. As the coil is flat, the projection of this edge on the curved surface of the head is prone to errors from visual parallax, both at times of target demarcation and each time the coil is placed for subsequent treatments. Additionally, one must rely on visual estimation to determine if the coil is tangentially placed on the scalp and at 45 degrees from the mid-sagittal plane. All of these potential sources of error can result in significant variability in coil placement between TMS operators and over repeated treatment session and, thus, may result in suboptimal therapeutic response.

For the treatment of major depression, the MS coil must be properly positioned for the duration of the treatment, which typically lasts from a few minutes to an hour. Current devices do not provide continuous feedback to the operator regarding proper coil placement. Small movements of the patient's head (for example, by head turning, coughing, sneezing, or shifting in the treatment chair) may change the coil-to-target placement and thereby impact clinical efficacy. Thus, provision of feedback on coil positioning to the TMS operator continuously over the duration of each treatment session may improve efficacy of the coil.

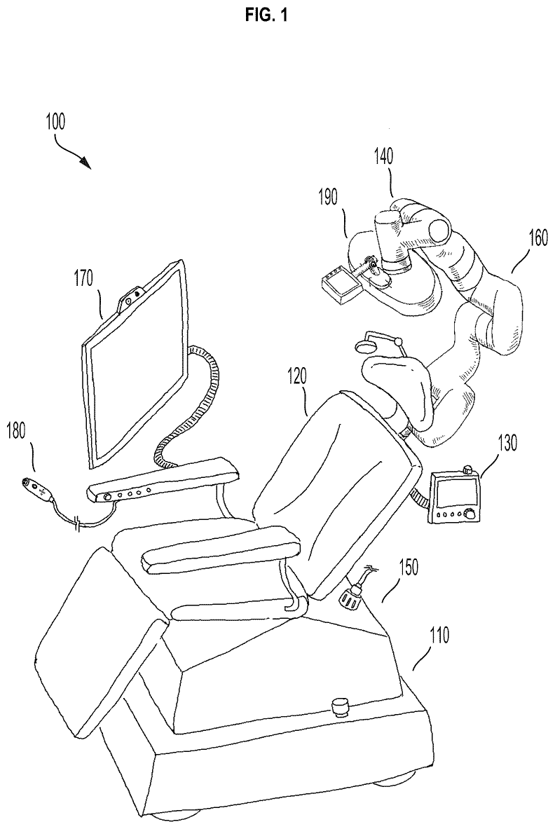

FIG. 1 is a depiction of an example TMS system 100 in accordance with embodiments described herein. The system 100 may include a base unit and primary computing module 110, a chair 120, an auxiliary controller 130, a TMS coil 140, a cable 150, an arm 160, a graphical interface console 170, a handheld controller/safety monitor 180, and a machine vision navigation device 190.

The base unit and primary computing module 110 may include a base unit and a primary computing module. The base unit may include a base housing to accommodate a TMS pulse generator. The TMS pulse generator may include a power supply, capacitor bank, a high-voltage switch, etc., controlled by the primary computing module. When the arm 160 implements a robotic arm to hold the TMS coil 140, the power supply and control hardware for the arm 160 may be housed within the base unit. To ensure stability, these heavier components may be placed at the bottom of the base unit. The base unit may also feature lockable wheels for ease of transportation, and/or may include an emergency shutoff switch located on each side of the base unit. In some examples, the base unit and primary computing module 110 may be located a module outside of the chair 120.

The primary computing module may sit atop the base unit and may include a primary computing device (e.g., a microcontroller and/or computer that controls the pulse generator and the arm 160, and communicates with various sensors, displays, and additional computing devices in the system 100), one or more wireless transceivers (e.g., Bluetooth radio, WiFi, long-range radio, 2.4 GHz radio, cellular, or any combination thereof), a global positioning system (GPS) module, storage or memory (e.g., SD card, EEPROM, SRAM chip, or other methods to locally record information on each TMS treatment session in non-volatile storage, potentially used in conjunction with a cloud-based platform), a real-time clock (RTC) to track current time and timepoints of recordings, a microphone (e.g., to allow a patient to interact with remote TMS operators, physicians, or other clinical providers, technical support, or any combination thereof), a speaker for audible alarms and communication with clinicians or support, one or more access ports (e.g., for connecting additional sensors, controllers, human interface devices, for reprogramming, or any combination thereof), or any combination thereof.

The chair 120 may resemble a conventional medical chair and may be positioned on top of the base unit of the base unit and primary computing module 110. The chair 120 may include adjustable armrests and neck support. The armrests may include controls that allow for power-adjusted chair positioning. The head support configuration can be adjusted using two hinges, and its height can also be modified as needed. A sidebar attached to the head support may provide counterpressure on the opposite side of the head where the TMS coil is to be placed.

The auxiliary controller 130 may be easily accessed by the clinician, although these controls can also be found on the graphical interface console 170. The auxiliary controller 130 may include an emergency shutoff switch to disable the stimulator and the arm 160 (when a robotic arm is utilized), as well as hardware controls to start, pause, and resume treatments. Additionally, the auxiliary controller 130 may include a display that indicates system status and key treatment parameters such as stimulation frequency, intensity, pulse train duration, inter-train interval, treatment duration, pulses delivered, and treatment number.

The TMS coil 140 may be connected to the TMS pulse generator in the base unit and primary computing module 110 by a long, flexible wire. The TMS coil 140 may include a figure-of-eight winding pattern. However, circular or parabolic coil configurations may also be implemented for use in the system 100. In some examples, the TMS coil 140 may include an alignment device configured to align the TMS coil 140 with the desired target. In some examples, the TMS coil 140 may include sensors and actuators (e.g., incorporated into the TMS coil 140 or mounted on the bottom surface of the TMS coil 140) to aid in the accurate and reliable alignment of the TMS coil 140 with respect to the desired target. The alignment device may include one or more of the following, a contact sensor or array of sensors that detects presence of coil contact with the target and relative position of the target with respect to the center of the TMS coil 140 (under which is the area of maximal stimulation); one or more proximity sensors on the underside of the TMS coil 140 used to determine distance of the TMS coil 140 to the underlying surface or, when there is more than one, can determine if the coil is tangentially oriented with respect to the target surface; a 9-degree of freedom inertial monitoring unit (e.g., triple-axis accelerometer, gyroscope, and magnetometer), used to determine absolute orientation of the TMS coil 140 (e.g., it, may help to rotate the TMS coil 140 to a desired angle with respect to the mid-sagittal plane (e.g., 45 degrees) or other reference); one or more laser diodes that project lines onto the underlying surface along the vertical, horizontal, or both axes of the TMS coil 140, which may serve as helpful aids in the case of manual or automated coil positioning, and one or more temperature sensors to determine coil and ambient temperatures. In some examples, determination that a coil temperature satisfies a threshold (e.g., is greater than a certain cutoff) may trigger a safety shutoff of pulses until the coil temperature returns to a suitable temperature.

The cable 150 may connect the pulse generator of the base unit and primary computing module 110 to the TMS coil 140. The portion connected to the TMS coil 140 is not shown. The arm 160 may hold the TMS coil 140. The arm 160 may be a robotic mechanical arm or non-robotic mechanical arm. The example using the robotic arm may facilitate automated alignment of the TMS coil 140 with the desired target and compensation for head movements during treatment based on feedback from the navigation device and/or feedback from the components from the alignment device. The robotic version of the arm 160 arm may be manually positioned by the operator during treatment planning or when manually reconfiguring the coil. In some examples, the arm 160 may include at least 6 degrees of freedom (DoF) with the TMS coil 140 secured to the end-effector to permit flexible positioning and orientation of the TMS coil 140.

In the implementation where the arm 160 is a mechanical arm, the arm 160 may include a series of interconnected joints, with at least 6 DoF, and an end effector attached to the TMS coil 140. The joints may have demarcations to permit repeatable configuration and a mechanism to lock the joints into place. The base joint may permit axial rotation of the arm and can be set to lock into place when internally rotated towards the patient by a preset angle determined by the clinician during treatment planning. A torsion spring mechanism may be built into the base joint. The spring may provide a slight “give” when the arm 160 is rotated in either direction around the locked position, providing a gentle counterforce of the arm 160 against the patient's head and aiding in steady contact. The rotational joint may be locked into place using a solenoid or other electromechanical actuator that is normally open.

The graphical interface console 170 may be attached to the chair 120 with an adjustable arm, The graphical interface console 170 may face the patient during operation. The graphical interface console 170 may include a display. In some examples, the display includes touchscreen display. In some examples, the chair 120 may be driven by a microcontroller, computer, or a commercially available tablet, running a custom application, and connected via Bluetooth or other wireless methods to control and communicate with the navigation device, other peripherals such as the handheld controller, and primary computing device in the base unit. In some examples, the graphical interface console 170 may indicate one or more of power status, stimulation parameters, treatment progress, quality of coil contact with the target, and other fields such as time of day, day/date, time left in treatment, and time until the next pulse (in the case of rTMS protocols with pulse trains). In some examples, alerts or instructions for the patient can be displayed on the graphical interface console 170, as well as safety prompts and questions requiring patient response before and during treatment. The graphical interface console 170 may be configured to receive user input for relevant self-report questionnaire responses (e.g., measuring depression, PTSD, anxiety symptoms, sleep measures, quality of life, suicidality, pain severity, or other clinical ratings to track progress over the course of rTMS treatments).

The graphical interface console 170 may include software or hardware controls to operate the system 100, allowing a user (e.g., patient, caregiver, clinician) to start, pause, or stop a treatment, adjust stimulation intensity, and modify other device settings (as permitted by the prescribing clinician). For example, the graphical interface console 170 may enable self-titration of stimulation dose up to the therapeutic intensity programmed by the clinician or follow a pre-loaded titration protocol, but may prevent stimulation greater than the prescribed intensity (e.g., 120% of motor threshold). These controls may also be accessed through the auxiliary controller 130. The graphical interface console 170 may further include a camera facing the patient. In some examples, the camera may be used for communication with a remote clinician or technician. The camera stream (e.g., of video and/or successive still images) may be processed by a microcontroller as part of a machine vision system with head and eye/gaze tracking capabilities. The system 100 may detect excessive head movements, prolonged eye closure, or improper seating. The camera may serve as a “mirror” to assist the patient with cap placement prior to reach treatment session. The graphical interface console 170 may further include a gooseneck arm in which the data and power lines of the device can be passed through. Additionally or alternatively, the graphical interface console 170 may be battery powered.

The handheld controller/safety monitor 180 may allow for safety monitoring and another, immediately accessible method for the patient to control the system 100 (such as starting, stopping, or pausing the treatment). Multiple sensors can be contained within the handheld controller/safety monitor 180 to allow for safety monitoring, such as pulse oximeter, inertial monitoring unit to detect aberrant movements (such as might occur during a seizure), capacitive touch sensors or force sensing resistors (to ensure the unit is being held by the patient), or any combination thereof. In some examples, the handheld controller/safety monitor 180 may also include a fingerprint scanner for user authentication prior to initiating each treatment session. During treatment, the handheld controller/safety monitor 180 may be held in the hand opposite the brain hemisphere being stimulated, as motor activity with pulses suggests that the TMS coil 140 may be stimulating over the motor cortex (and should trigger an alarm if the intended target is the dorsolateral prefrontal cortex, as with depression).

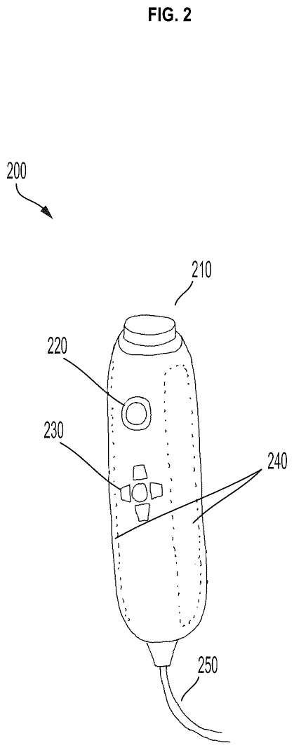

FIG. 2 is a depiction of an example controller 200 permitting patient control of a TMS system and for safety monitoring in accordance with embodiments described herein. In some examples, the handheld controller/safety monitor 180 of FIG. 1 may implement the controller 200. The controller 200 may include a switch 210 that may be configured to pause the treatment and, if pressed again, or held for a certain duration, can disarm the robotic arm and stimulator, and cause the arm to retract. In the case of using a mechanical arm, the lock at the base joint can disengage to permit the TMS coil and arm to rotate away from the patient. The switch 210 may be translucent to permit light from an underlying RGB LED to pass through. The controller 200 may further include another switch 220 that may be activated to start or resume a treatment. Optionally, additional switches 230 on the body of the controller 200 can be used to access other features, such as stimulation titration or to navigate the menu on the graphical interface. The additional switches 230 may include tactile or capacitive touch switches. The controller 200 may further include one or more of either capacitive touch sensors or force sensing resistors 240 to determine patient grip. For example, voltage of the force sensing resistor outputs or the raw readings from the capacitive electrodes could be read by the microcontroller contained within the controller 200 and used to determine if the patient is holding the controller 200 and the relative magnitude of the grip. The controller 200 may further include sensors including temperature sensor, an inertial monitoring unit (IMU) that is continuously monitored during operation, vibratory motor (eccentric rotating mass (ERM) or linear resonant actuator (LRA)), and wireless transceiver. In some examples, the controller 200 may include a fingerprint scanner (not shown, on the back of the device) for user authentication. The controller 200 may be wired (c), in which case the wire would contain power rails and optionally, data lines to communicate with other peripheral devices or the master computing module. In the wireless version, a rechargeable battery and charging circuitry could be contained within the controller 200.

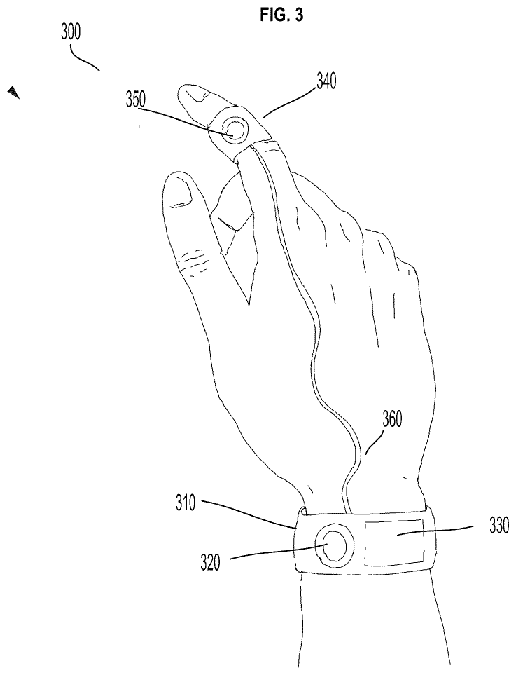

FIG. 3 is a depiction of an example wrist controller 300 permitting patient control of a TMS system and for safety monitoring in accordance with embodiments described herein. In some examples, the handheld controller/safety monitor 180 of FIG. 1 may implement the wrist controller 300. The wrist controller 300 may include a wireless wrist controller 310 that is worn on the side contralateral to the brain hemisphere being stimulated. The wireless wrist controller 310 may be connected wirelessly to the master computing device and peripherals. The wireless wrist controller 310 may be is constructed with some of the components on a flexible PCB, and may be secured by a hook and loop strap with an clastic component to provide light tension. The wireless wrist controller 310 may include additional components not visible in this figure, such as a microcontroller with wireless capabilities (e.g., the ESP32 series); a rechargeable battery and charging circuit; temperature sensor; pulse oximeter; heart rate sensor; an IMU with at least 3 axis accelerometer and 3-axis gyroscope; and method of haptic feedback (e.g., a LRA). A tactile or capacitive touch switch, or force sensitive resistor 320 may allow direct control of the TMS system. The 320 may function in the same manner as the switch 210 of FIG. 2. The wireless wrist controller 310 may further include another switch, similar to the switch 220 of FIG. 2, that can be used to start or resume a treatment. An optional display (e.g., a flexible organic light-emitting diode (OLED) display) 330 may be incorporated in the wireless wrist controller 310 to indicate information such as treatment status and system warnings or errors.

In some examples, the wrist controller 300 may include an attached fingertip controller and sensor module 340. The fingertip controller and sensor module 340 may be incorporated into an clastic silicone sleeve, or with a hook and loop strap with clastic component, as with the wireless wrist controller 310. In the example of the wrist controller 300 using the fingertip controller and sensor module 340, the temperature sensor, pulse oximeter, heart rate sensor, and IMU could be placed within, on a flexible PCB substrate. The fingertip controller and sensor module 340 may include a switch 350, as with the wireless wrist controller 310, which the user can actuate to pause or stop the treatment, and another (not shown) that can be used to start or resume the treatment. A flexible connector 350 may attach the fingertip controller and sensor module 340 to the wireless wrist controller 310. In this example, the wires contain power and data lines to communicate with the microcontroller in the wireless wrist controller 310.

Turning back to FIG. 1, the machine vision navigation device 190 may be a compact, portable device can be used as an alternative to, or in addition to, the alignment device of the TMS coil 140. The machine vision navigation device 190 may be battery-powered, self-contained, and easily retrofitted onto existing ones of the TMS coil 140. The machine vision navigation device 190 may optical neuronavigation in a more compact form than existing solutions. The machine vision navigation device 190 may be used as part of the system 100 for automated coil alignment, or independently of the system 100.

The machine vision navigation device 190 may include a camera, a microcontroller (e.g., as part of a machine vision system), a graphical display showing the camera stream (e.g., of successive still images and/or video) and information regarding the location and orientation of the fiducial(s) relative to the TMS coil 140, or any combination thereof. The display may show status information such as battery life, error messages, and instructions or graphics for manual alignment of the coil to a desired target configuration (e.g., over the treatment site or motor hotspot). The machine vision navigation device 190 may also include a rechargeable battery, RTC, touchscreen, tactile switches, EEPROM, microSD card or other non-volatile storage, a wireless transceiver for communication with another computing device (e.g., other peripherals and the primary computing device), or any combination thereof. The information may be displayed on the graphical display (or auxiliary controller 130 or the graphical interface console 170) and used to control the arm 160 or aid in manual alignment of the TMS coil 140.

The microcontroller of the machine vision navigation device 190 may employ machine vision to identify the position, orientation, and ID of the visual fiducial markers, enabling repeatable alignment of the TMS coil 140 with respect to the treatment target, motor hotspot, or other desired pose. Note that the term “pose” refers to a particular location and orientation relative to the camera and the TMS coil 140, while “configuration” refers to the position of each joint of the arm 160.

The navigation device could permit panning and tilting of the camera portion. The joints for pan and tilting have markings for consistent reconfiguration and detents for repeatability. The joints can be locked to prevent accidental displacement. The navigation device can be attached to the TMS coil 140 using snap fit, screws, magnets, or other methods allowing for removal and replacement. Alignment markings on the base of the navigation unit aid in consistent mounting to the TMS coil 140.

In contrast to conventional neuronavigation systems, which often require complex setups, ample space, associated brain MRIs, and anatomical registration, the navigation device described here may be portable, low-cost, and compatible with existing TMS systems, making it suitable for routine clinical use and across various TMS machines.

In another embodiment, the camera and machine vision system may be mounted from a support arm extending from the chair, above the head, with a field of view that includes tags placed on the TMS coil 140 and the patient's head. In this setup, the “target” configuration consists of the difference in poses between the coil and the fiducial marker, as assessed by the machine vision system. The machine vision navigation device 190 may provide superior accuracy and repeatability to existing technologies/methods, and offers improved performance, with the optical method providing greater precision.

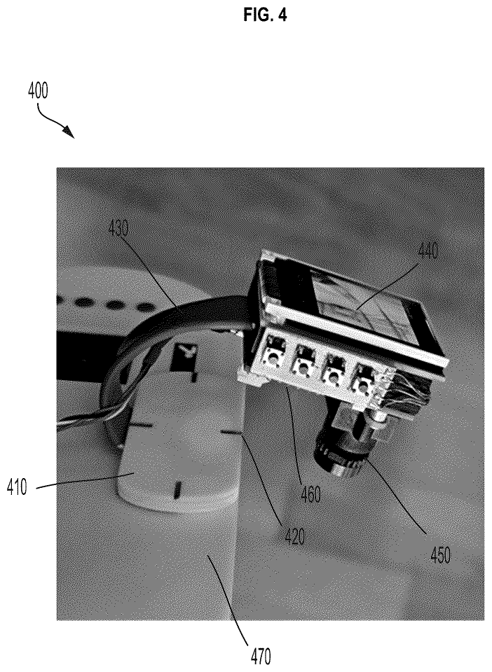

FIG. 4 is a depiction of an example machine vision navigation device 400 retrofitted onto a previously constructed prototype TMS coil with an alignment device in accordance with embodiments described herein. In some examples, the machine vision navigation device 190 of FIG. 1 may implement the machine vision navigation device 400. The machine vision navigation device 400 may include a base plate 410. In some examples, the base plate 410 may be translucent to allow a LED of the TMS coil 470 (e.g., the TMS coil 140 of FIG. 1) to remain visible. The machine vision navigation device 400 may further include markings 420 along the edge of the base plate 410 to aid in manual alignment of the machine vision navigation device 400 to the TMS coil 470. The machine vision navigation device 400 may further include a mounting arm 430 with a semiflexible neck that support the main body of the machine vision navigation device 400, although a rigid arm, with or without adjustable joints, may also be used. The machine vision navigation device 400 may further include a graphic display 440 for manual alignment of the TMS coil 470 and/or a camera 450 as part of a machine vision system used by the device's computing unit. The machine vision navigation device 400 may further include tactile switches or touch switches 460 that can be used to operate the machine vision navigation device 400. Capacitive touch switches may be preferable because they require negligible force to activate, which is less likely to disturb the mounting arm 430 of the machine vision navigation device 400 when operated.

In this example, the switches of the machine vision navigation device 400 may trigger the following functions: recording a pose, toggling between patients and treatment/hotspot settings, controlling a forward-facing LED to illuminate the underlying surface, and changing modes of operation from recording poses to showing a “find” view, where information is displayed to allow for manual realignment of the coil to a stored treatment target or hotspot pose. The machine vision navigation device 400 may be powered by USB, could also be powered by a rechargeable battery. Additionally, the optical and electronic components of the machine vision navigation device 400 (e.g., the graphic display 440, the camera 450, and/or the tactile switches or touch switches 460) could be made more compact using a smaller development board.

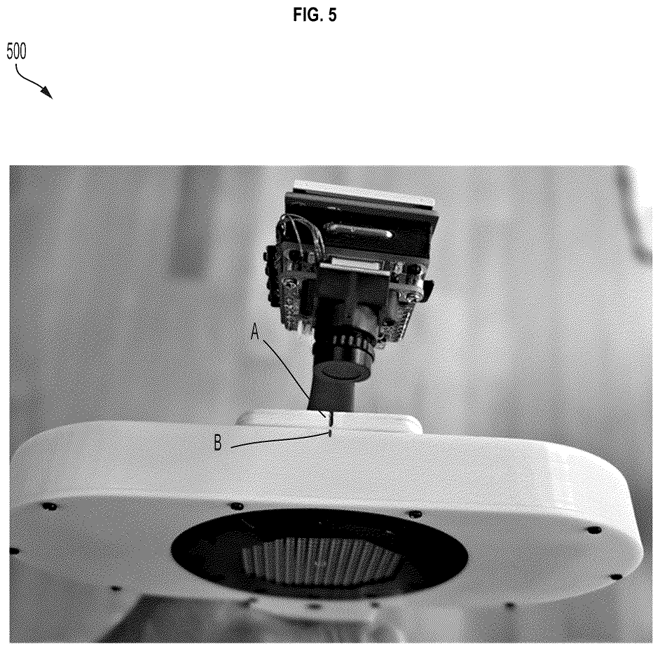

FIG. 5 is a depiction of the example machine vision navigation device 400 of FIG. 4 of the alignment markings on the base plate 410 and the coupled TMS coil 470 in accordance with embodiments described herein. In some examples, the machine vision navigation device 400 may include a module that can be securely fixed to the 470 and permit snap-fitting of the machine vision navigation device 400.

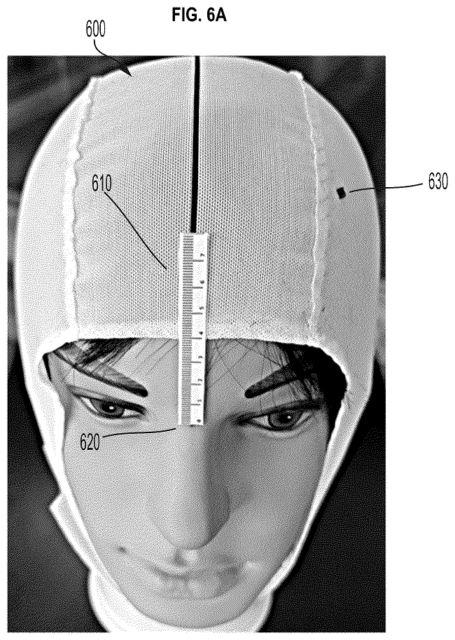

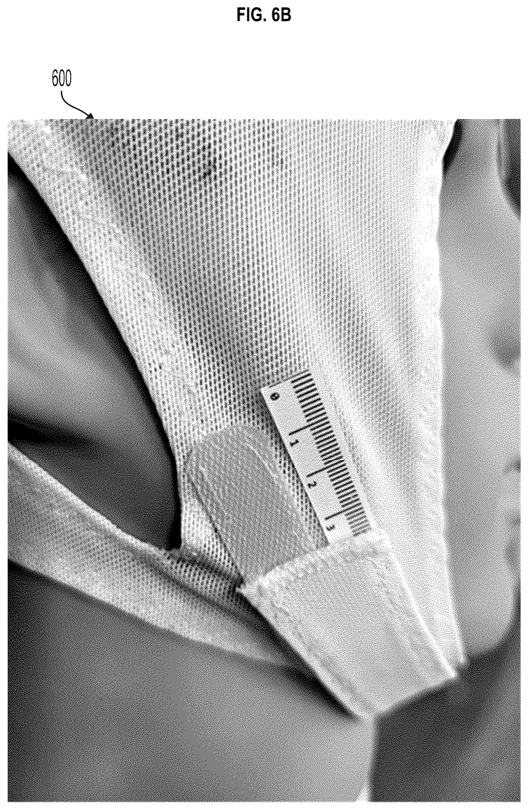

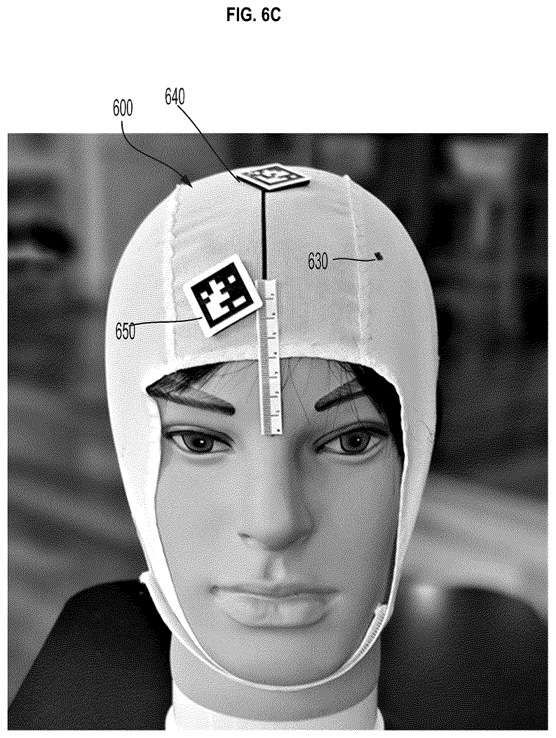

To facilitate alignment of the TMS coil, a form-fitting cap may be worn by the patient. The cap may have a chin strap and a reference line along the mid-sagittal plane, to aid in centering the cap on the head. A flexible guide with ruler markings can be affixed to the cap, such that the ruler terminates at the nasion. The distance from the nasion to the cap edge will vary patient-to-patient. An additional ruler marker is permanently fixed to the chin strap, such that the snugness of the cap can be consistent between sessions. FIGS. 6A-6C depict various view of an example implementation of a cap 600 in accordance with embodiments described herein.

FIG. 6A depicts a first view of the cap 600 on a head of a patient in accordance with embodiments described herein. During the treatment planning session, a ruler 610 may be affixed vertically to the cap 600 such that it extends to the nasion 620. When making subsequent fittings, the cap 600 should be adjusted so that the ruler aligns with the nasion 620 again. Additionally, an optional conductive marker 630 may be utilized to identify the treatment location. In this case, the treatment target is the dorsolateral prefrontal cortex or F3 on an EEG coordinate system, as per the protocol for treatment of major depressive disorder. The conductive marker 630 may be used in combination with a coil alignment apparatus to facilitate the placement of fiducial markers and the recording of the desired pose at the time of treatment planning. This conductive marker 630 may be distinct from a fiducial marker and can be used to indicate treatment target or motor hotspot locations, when used in conjunction with a TMS alignment apparatus. The conductive marker 630 may include conductive material (e.g., metal foil; conductive fabric, felt, or foam; Velostat; conductive ink; etc.), allowing it to electrically couple with the contact sensor grid on the underside of the TMS coil or act as a plate of a capacitor if used with capacitive touch method of contact sensing.

FIG. 6B depicts a side view of the cap 600 on a head of a patient in accordance with embodiments described herein. As shown in FIG. 6B, another ruler may be permanently attached to the cap 600. The ruler marking at which the chin strap terminates is recorded and reproduced during subsequent fittings. This ensures consistent cap placement across multiple sessions. In the example fitting depicted in FIG. 6B, the chin strap ends of the cap 600 at the 3.6 cm marking. A refined prototype would have a hook fastener along both lengths of the ruler.

FIG. 6C depicts a third view of the cap 600 on a head of a patient in accordance with embodiments described herein. The view of the cap 600 depicted in FIG. 6C is after demarcation of treatment target and motor hotspot, as conducted during treatment planning. That is, FIG. 6C depicts an example of the cap 600 with affixed fiducial markers during treatment planning to facilitate alignment of the TMS coil over the motor hotspot (aligned with fiducial marker 640) and the treatment target (aligned with marker 650). The navigation device shows that when the TMS coil is optimally realigned over the target (630) (over F3), target marker 450 is in the desired pose for the treatment. The fiducial markers 640 and 650 may be are affixed to a rigid substrate with adhesive backing to maintain their position across multiple sessions.

To further enhance the stability of the markers 640 and 650, the cap 600 may have a low profile loop texture on its outer surface, while the fiducial markers have a hook texture on their bottom surface. This design may facilitate stable alignment across repeated sessions while permitting repositioning if needed.

Unlike the conductive marker 630, the fiducial markers 640 and 650 may include a visual tag recognizable by the machine vision algorithm utilized by the navigation device, providing information on the 3-dimensional location and orientation (e.g., pose) of the tag relative to the TMS coil. In this example, an AprilTag 2 is shown, but other types of fiducial tags enabling pose estimation can also be employed. The method may allow for reliable alignment of the TMS coil with respect to a target post as programmed by the clinician.

In the case of AprilTag 2, there are 587 unique tag IDs. For this TMS system, certain IDs or ranges of IDs can be allocated for specific purposes, such as marking the treatment target, motor hotspot, coil homing location, camera calibration, self-testing, and for safety or error purposes (e.g., triggering disarming the stimulator, robotic arm withdrawal, safety notification, etc., when a particular fiducial marker in the field of view of the camera). In the case of applying magnetic stimulation to other parts of the body, such as for peripheral nerve stimulation requiring repeated sessions, one could consider applying a temporary tattoo of fiducial markers in the field of view of the camera (e.g., by analyzing successive still images and/or video from the camera) when the coil is over the target and record it as a reference configuration for subsequent coil realignment.

In some examples, the system 100 of FIG. 1 may be equipped with a laser that projects a line along the midline of the chair 120 and headrest. This laser may assists with the manual alignment of the cap 600 and the patient's head relative to the head support and the chair 120. A camera facing the patient, incorporated into the graphical interface console 170, may aid in self-fitting of the cap 600.

Turning back to FIG. 1, the system 100 may employ user authentication to enable system operation by the intended patient and retrieve appropriate settings, including stimulation parameters, for that individual. Settings can include settings of the chair 120, joint position in the case of the arm 160, desired coil orientation, and treatment parameters like stimulation intensity, pulse frequency, pulses per session, number of pulse trains, interval between trains, treatment number, date/time of previous treatment, etc. Authentication codes may be entered through the auxiliary controller 130 or the graphical interface console 170. Such codes may be sent to the patient via URL link through email, SMS, or other methods. Use of a fingerprint scanner, NFC card, PIV/ID card, QR code scan, facial recognition using the camera, login/password, or other existing methods could be used for authentication. If a clinician or other system administrator unlocks the system 100, it may permit access to broader controls, such as adjustment of treatment parameters and target coil configuration.

In some examples, the system 100 may further include any combination of a cellular radio, radio transceiver, Bluetooth, WiFi, long-range radio (LoRa), or other wireless modality for communications. These methods may be used to allow secure two-way communication, including syncing of data from the patient's phone, the remote TMS system, a clinician's office, or a web server with the appropriate stimulation parameters for that patient. The system 100 may have the capability to upload treatment data to the cloud for retrieval and review by the prescribing physician or other clinician, and could also be viewed by the patient for self-tracking of clinical progress through a TMS course.

The wireless devices may also be used for local communication between parts of the system 100, including with the master computing device, and with external systems through the internet or other wireless protocols. There are many possible configurations for a wireless setup, but in the proposed example, the same wireless transceiver type may be utilized in all the modules of the system 100, including the graphical interface console 170, the auxiliary controller 130, the handheld controller/safety monitor 180, the machine vision navigation device 190, the chair 120, and the base unit and primary computing module 110. In a specific, non-limiting example, the system 100 may include the ESP32 microcontroller series from Esprissif Systems. In this example, the ESP-NOW protocol (open-source communication protocol by Espressif Systems) is used, as it permits wireless, low-power and low-latency communication between ESP32 microcontrollers, without the need for an access point or router. The ESP32 also has Bluetooth, so that it can communicate with peripheral devices like the patient's cell phone. In the example system, the primary computing module and the graphical interface modules also connect to external Wi-Fi networks using the same ESP32 microcontrollers, to support access to the internet to upload data and permit remote operation and video communication. The microcontroller in the base unit and primary computing module 110 may also be associated with a GPS module and multiple wireless transceivers, including a cellular radio, and optionally 2.4 GHz radio and long-range radio. The modular nature of this setup permits components to function without external wiring, or if desired, limiting wired connections to power rails, though rechargeable batteries could also be used to power the peripheral devices. Having multiple microcontrollers/computing devices can improve the efficiency and responsiveness of the system 100. The base unit and primary computing module 110 which controls the robotic arm may be equipped with a more powerful computing device, such as a single board computer.

A clinician may determine the motor “hotspot” and motor threshold using conventional methods and mark the target location. For treating depression, the left dorsolateral prefrontal cortex (DLPFC) is typically targeted, which can be estimated using the F3 location on the 10-20 EEG coordinate system (e.g., the BeamF3 method). The TMS coil 140 used by the operator may include an alignment apparatus that can facilitate determination of the proper position and orientation of the coil with respect to the target.

While the TMS coil 140 and the arm 160 are in the desired configuration (e.g., such that the TMS coil 140 is positioned over the target [F3], rotated 45 degrees with respect to the mid-sagittal line, and oriented tangentially to the underlying surface), the operator may affix a fiducial marker (e.g., the fiducial marker 640 or 650 of FIG. 6C) on the patient's cap. The absolute position and orientation of the marker is not critical, as long as it is placed near the center of the navigation device camera's field of view. The operator can actuate the “record” switch (switch 460 of FIG. 4) when the TMS coil 140 is in the desired configuration. This triggers the microcontroller on the machine vision navigation device 190 to store variables into the local file system including date, time, fiducial marker ID number, and pose estimate (x, y, z, position, and rotation along x, y, and z axes) of the camera of the graphical interface console 170/the TMS coil 140 compared to the fiducial marker. A still image of the camera feed can be associated with the pose data. The data could be also be stored in a microSD card or other medium, and transmitted wirelessly to the primary and peripheral devices in the system 100. The system 100 may associate these variables to the unique patient as previously determined during the authentication procedure, or as manually selected by the clinician. These stored variables serve as reference values to permit re-alignment at subsequent timepoints.

The same approach can be applied to record coil configuration when stimulating over the motor hotspot to measure the motor threshold. Another example of a fiducial marker could consist of a group of two or more markers arranged in a known relative configuration. This would increase the accuracy of relative pose estimation, as grouping the markers would smooth out errors from pose estimation over multiple fiducial markers.

When the machine vision navigation device 190 is used as part of the system 100 with a robotic arm 160, the angle at each joint of the mounting arm 160 is recorded by the master computing system when the “record” switch is activated. If a mechanical arm 160 is used, the joint angles are recorded manually by the clinician. The base joint, with axial rotation, can be rotated internally from the target configuration by a few degrees, as set by the clinician, such that when the locking mechanism is engaged, the TMS coil will apply a gentle pressure against the patient's head and ensure steady contact with the target.

Each unique target configuration (e.g., motor hotspot and treatment locations) per patient will be associated with corresponding support arm joint angles, chair settings, and coil pose, to allow for consistent reproduction over time. The data may be stored in local non-volatile storage, a network device, or cloud-based platform.

When the machine vision navigation device 190 is used as part of the system 100, real time relative pose data can be streamed wirelessly to the master computing system, allowing automated closed-loop movement of the robotic arm to consistently realign the TMS coil 140 to a target.

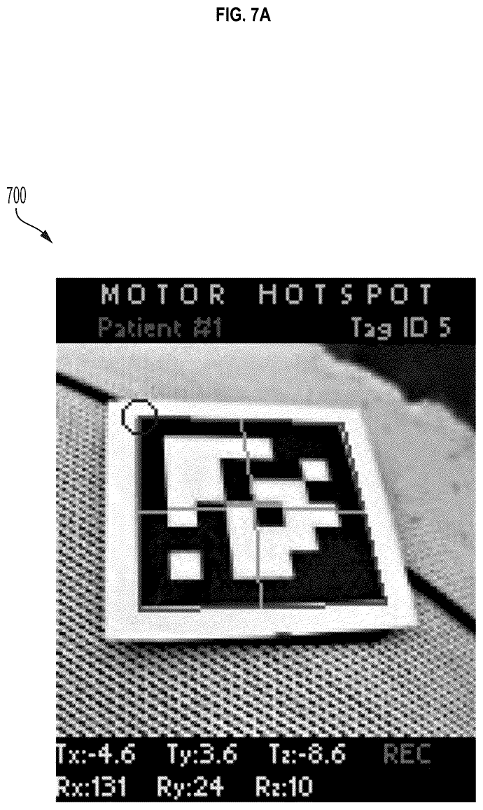

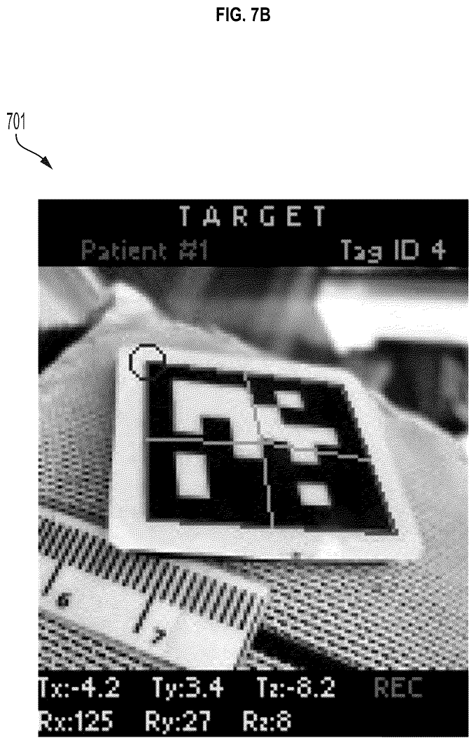

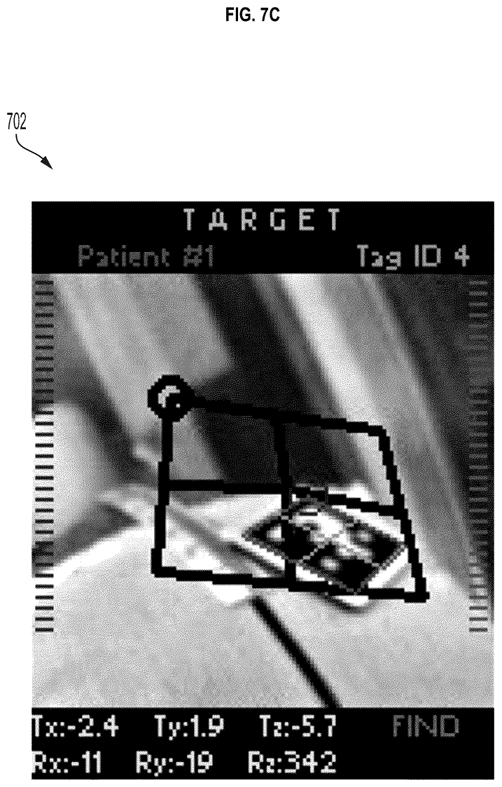

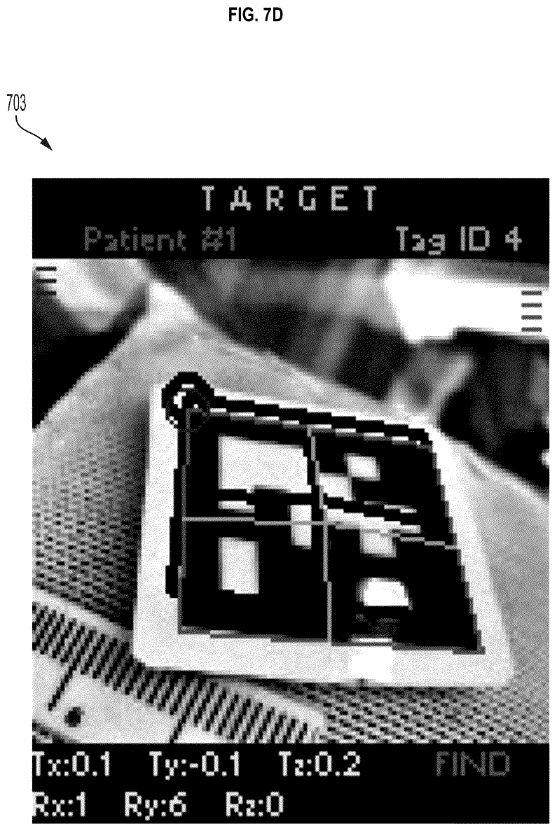

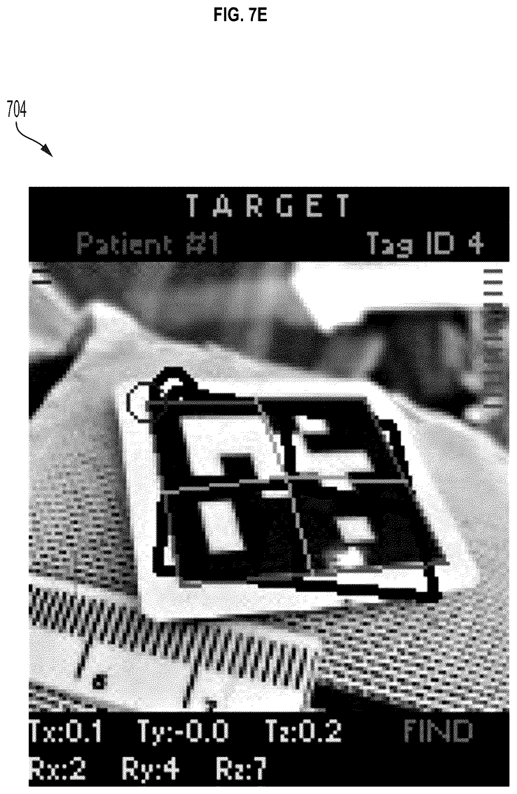

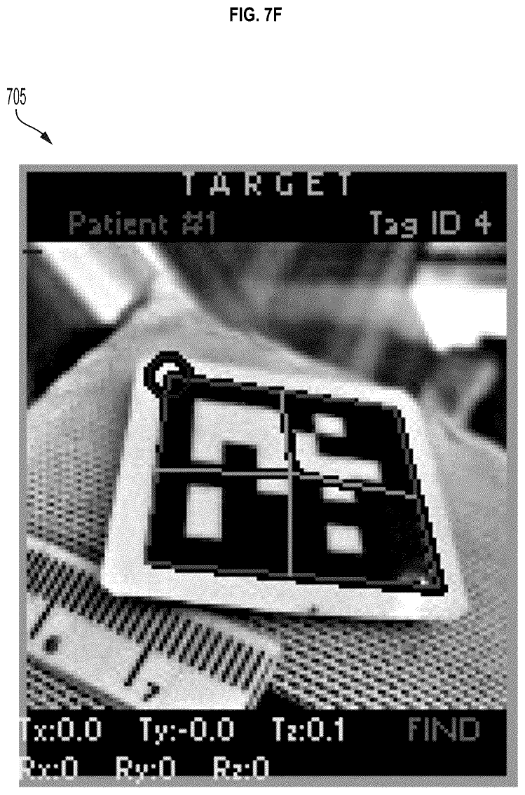

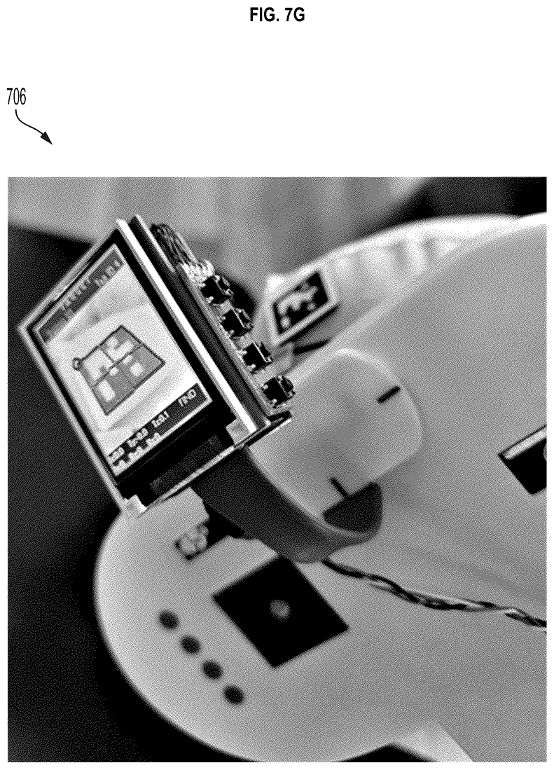

FIGS. 7A-7F depict various images of a camera feeds after processing by a machine vision algorithm in accordance with embodiments described herein. A resolution of the camera and display may affect accuracy and precious of the navigation function of the machine vision navigation device 190, in some examples. With reference the image 700 of FIG. 7A, on the top portion of the display is an indication as to whether the machine vision navigation device 190 is detecting a fiducial marker designated for treatment target, motor hotspot, or other purpose; patient identifier; and fiducial marker ID number. In some examples, this portion could also include the patient's name or other identifier may be shown. On the main portion of the display is the camera feed with an overlay highlighting the fiducial marker's border, center, and one corner. The bottom portion of the display shows the pose of the fiducial marker relative to the TMS coil 140, displaying translation in x, y, and z coordinates (e.g., in arbitrary units) and rotation about the x, y, and z axes e.g., roll, pitch, and yaw) in degrees. The “REC” indicator may indicate that the machine vision navigation device 190 is record mode, allowing an operator to capture a coil pose associated with a motor hotspot or treatment target when actuating the “record” switch (e.g., the switch 460 of FIG. 4). The image 701 of FIG. 7B depicts the display on the machine vision navigation device 190 when the TMS coil 140 is situated over the treatment target.

For subsequent coil alignment to a previously recorded state, as required before each treatment session, the machine vision navigation device 190 may be set to “find” mode, allowing the operator to select a previous target state. The machine vision navigation device 190 may then calculates the differences in each of the six degrees between the camera and fiducial pose compared to the desired configuration. Thus, when the TMS coil 140 is optimally reconfigured to match alignment with a recorded state, the differences in the six degrees will be zero or within an acceptable tolerance.

With manual realignment, the operator can use guidance from the machine vision navigation device 190, which can display written instructions and/or graphical indicators to assist in aligning the TMS coil 140 in six degrees (e.g., relative position in x, y, and z coordinates, and roll, pitch, and yaw) with respect to the target configuration. In one example, a thick line drawing of the previous fiducial is may be overlayed on the display of the machine vision navigation device 190. Audible, visual, and haptic feedback may be provided by the machine vision navigation device 190 to indicate when the TMS coil 140 is positioned within a certain tolerance from the desired target pose, or misaligned beyond a certain threshold. The images 702-705 of FIGS. 7C-7F depict examples of how the machine vision navigation device 190 can be used to manually realign the TMS coil 140 to a target pose.

The image 702 of FIG. 7C depicts the display on the machine vision navigation device 190 when the TMS coil 140 is above the head and grossly misaligned. The image 702 illustrates the display when the navigation device is in “find” mode, enabling the operator to manually realign the TMS coil 140 to a previously recorded state, such as over the treatment target or motor hotspot. The fiducial marker ID appears in the upper right portion of the display, shown in green if it matches the recorded fiducial marker ID. If the fiducial marker ID does not match the recorded value, it is displayed in red along with an error message and alarm which may be audible, haptic, or visible through a flashing LED.

The middle section presents the camera feed with the fiducial marker features highlighted, similar to that shown in the images 700 and 701. Additionally, a thickened line drawing of the previously recorded fiducial marker in its target pose is included. In this example, the treatment target fiducial marker for patient #1 is displayed. The bottom section reveals the delta values for translation and rotation in all three axes, compared to the stored fiducial marker's pose. When all values are zero, the TMS coil 140 has been realigned to a previously stored state. In this example, the TMS coil 140 is significantly misaligned with the target pose, as shown by the relatively large delta values for translation and rotation.

The display also features two segmented bar graphs on the left and right sides, representing the total error in translation (e.g., blue bars on the left) and rotation (e.g., red bars on the right) compared to the desired pose. In some examples, the display could provide a graphical depiction of the required coil movement to match the recorded pose. Alternatively, a 3D cube overlay on the current and target fiducial markers could convey this information in an intuitive manner. A secondary display may also be utilized, such as one depicting an artificial horizon, or a flight director altitude indicator (FDAI)-type display to indicate rotational deviations from the programmed state and aid in manual alignment of the TMS coil 140.

The image 703 of FIG. 7D is similar to the image 702, except that the TMS coil 140 is closer to the relative position and rotation of the recorded treatment target, as depicted by: smaller delta values on the bottom of the display, the current fiducial marker being close to the target reference fiducial marker line drawing, and the smaller error bar graphs. The image 704 of FIG. 7E is similar to the image 703 when the TMS coil is close to the target pose, except with a larger relative error in rotation as compared with translation. The image 705 of FIG. 7F is to previous images 703 and 704, but showing the TMS coil 140 to be in close alignment (“on target”) to the desired target pose for patient #1. In this case, there is a small error in translation (as depicted by the bar graph on the top left), and no rotational discrepancy from the target (as depicted by no error bars on the top right). Accordingly, the delta values on the bottom of the display are at or close to zero. A green border is shown when the coil is within an acceptable tolerance to the target state. An audible, haptic, or other type of alarm may also be used to indicate when the TMS coil 140 is aligned or misaligned.

FIG. 7G depicts an image 706 of the image 705 of FIG. 7E from a different view, in accordance with embodiments described herein. The image 706 shows the machine vision navigation device 190 mounted on the TMS coil 140 with an alignment apparatus. The machine vision navigation device 190 depicts the TMS coil 140 to be in the target pose. Accordingly, the TMS coil 140 with alignment apparatus shows that it is “on target”: making centered contact over the target marker, tangential to the surface, and rotated 45 degrees to the mid-sagittal line.

Turning back to FIG. 1, in the automated embodiment of the system 100, the output from the machine vision system (e.g., translation and rotation error in 3 axes could be fed into a control for a robotic arm of at least 6 degrees of freedom (DoF) using a proportional-integral-derivative (PID) controller to move the end effector appropriately to realign the TMS coil with respect to the target.

In the case of automated coil alignment using a robotic arm 160, the machine vision navigation device 190 may function as part of a closed-loop system to enable automatic reconfiguration of the coil consistently over the desired target. A robotic arm 160 with at least 6 degrees of freedom (DoF) could be used for this purpose.

The 100 may be configured to enable a clinician to manually move the TMS coil 140, either through passive motion or by using the machine's controls. This feature allows the clinician to adjust target configuration based on clinical indications. For instance, if a patient experiences scalp discomfort, rotating the TMS coil 140 a few degrees clockwise may alleviate or resolve the issue. By using the machine vision navigation device 190, the clinician can adjust the target configuration as needed on a per patient or per treatment level.

Before activating the stimulator and robotic arm, the system 100 may be unlocked through user authentication, which can be achieved using various methods such as QR code, fingerprint scan, PIV card, text code, facial recognition, etc. If a clinician enables the system 100, it permits adjustment of certain settings, such as treatment parameters, which may not be accessible to the patient. The clinician can select the patient being treated, and the system 100 may retrieve the data associated with that patient (e.g., prescribed treatment protocol, chair settings, arm configuration for motor threshold determination and treatment target). In cases where the patient uses the machine in an at-home setting, these data will be retrieved upon patient authentication. The data may be stored locally and in a cloud-based environment.

Before initiating a treatment session, the patient may complete a safety screening. Responses can be entered through the graphical interface console 170 facing the patient. Pre-treatment steps may include confirming the removal of jewelry or other metallic objects from the head and neck, ensuring the patient is using hearing protection, and screening for suicidal ideation. Some questions pertaining to potential seizure risk or alterations in motor threshold may be askes, including any medication changes since the last treatment and inquiring about changes in sleep patterns and alcohol consumption. Certain responses may trigger follow-up prompts.

For instance, if there has been a change in medication, the system 100 may ask the patient to specify the medication change. If the medication is known to affect seizure or motor threshold, the system may lock out, requiring a clinician operator to re-enable it, as motor threshold may need rechecking, which would impact the stimulation parameters needed for treatment. In cases where the patient reports suicidal ideation, additional questions to determine the severity, plan, and intent may be displayed. Certain responses indicating acute suicide risk could activate an emergency alert protocol, which may involve contacting a clinical provider or a suicide hotline. The system 100 may include a SIM-module to enable cellular connectivity. Once the safety screen is completed satisfactorily, the TMS coil 140 may be aligned over the treatment target before initiating treatment.

When not in use, the arm 160 may be positioned in a way that provides adequate clearance for the patient, allowing the patient to sit in or exit the chair 120 without colliding with the TMS coil 140 or the arm 160.

In cases where the system 100 utilizes a non-robotic mechanical arm, each joint can be adjusted to the recorded angles corresponding to the treatment target for the patient. The last joint to be set is the base joint of the arm, which is a rotational joint usually externally rotated away from the patient when the machine is not in use. After all other joint angles are set, the user can rotate the arm 160 internally until it contacts the patient's head with sufficient pressure, at which point the base joint locks into place. The base rotational joint may have a spring action to aid in maintaining steady contact with the patient's head after locking. The patient can manually release the arm 160 at any point using the handheld controller/safety monitor 180, or the graphical interface console 170, allowing the TMS coil 140 to return to its resting configuration.

In cases where a robotic arm is used, the TMS coil 140 may initially be positioned above the patient's head and centered over the target fiducial marker, based on input from the camera/machine vision system. Data from the machine vision system guides the TMS coil 140 to the target configuration, centering it over the target location and orienting it tangentially to the scalp. Specifically, the recorded pose information for the particular patient and target may be compared to the real-time reading from the machine vision navigation device 190. The target configuration is reached when the differences between the current and recorded pose elements are all zero or within a set tolerance (e.g., the TMS coil 140 is configured in the equivalent relative position [x, y, z] and rotational angles [roll, pitch, yaw] from the target configuration recorded by the clinician during the treatment planning phase).

Optionally, the arm 160 may follow a programmed sequence of movements recorded with manual placement by the clinician to approximate the desired configuration. Using closed-loop feedback from the machine vision system, the TMS coil 140 may be moved to the final target configuration, based on real-time feedback from the machine vision navigation device 190. Force and torque sensors incorporated into the end effector and joints of the arm 160 could be utilized to trigger arm withdrawal if excessive force is detected on the end effector or individual joints.

The system 100 may be operated by the clinician, patient, or a combination of both, depending on the treatment setting and clinical needs for the specific patient. Controls on the base unit and primary computing module 110, accessible to the clinician, can be used to configure and operate the machine in-person. These controls can also be accessed remotely.

The graphical interface console 170 facing the patient may provide another method for operating the system 100 by either the patient or clinician. Optionally, in an at-home setting, the patient may use the handheld controller/safety monitor 180 for machine operation and safety monitoring. The patient may operate switches on the handheld controller/safety monitor 180 to start/resume, pause, and stop the treatment.

Once the necessary conditions for user authentication, safety screening, and appropriate coil configuration are met, the treatment may be initiated, following the programmed simulation parameters. The patient can start, pause, and stop treatments using the graphical interface console 170 and/or the handheld controller/safety monitor 180. Certain conditions, such as error conditions or safety alerts, can trigger the treatment to pause or terminate.

During treatment, the system 100 may continuously monitor the patient through the handheld controller/safety monitor 180, machine vision modules incorporated into the graphical interface console 170 facing the patient, another camera on or above the TMS coil 140 used by the machine vision navigation device 190, or any combination thereof. If a robotic arm is used, the configuration of the arm 160 may adjust as needed to account for patient head movements and maintain optimal contact with the target, using feedback from the machine vision navigation device 190. With a mechanical arm, the machine vision navigation device 190 can signal an alarm to the user when the head is not suitably situated relative to the TMS coil 140, although only minor adjustments should be needed if the arm joints are preadjusted to the angles determined at the time of treatment planning.

Upon treatment completion, the TMS coil 140 may return to its resting configuration. If a robotic arm is used, the arm 160 may automatically retract from the patient's head and returns to its starting configuration. With a mechanical arm, treatment completion causes the lock securing the rotational joint at the base to disengage, so the arm 160 can rotate externally away from the patient's head.

Optionally, the system may prompt the patient to complete a questionnaire regarding side effects or other symptoms after the treatment is completed.

The system 100 may include several methods for monitoring the patient to assess safety and efficacy. The wireless capabilities and camera allow the potential for remote monitoring and operation. The system 100 can be operated in various modes, depending on the setting and clinical need, such as in-person by the clinician in the clinical or at-home setting, remotely operated by a clinician, patient-operated in an at-home setting, with the option of real-time clinician oversight or remote operator control in alarm conditions.

The system 100 may include a 6-axis force and torque sensor for collision detection. If the sensor reading exceeds a specific threshold, the arm 160 may retract. The individual joints in the arm 160 may also include torque sensors that trigger pausing if any of them exceed a certain threshold.

The system 100 may further include a pause/shut-off switch accessible on the handheld controller/safety monitor 180, the graphical interface console 170, on one or more sides of the base unit and primary computing module 110, the auxiliary controller 130, and remotely. The switch may disarms the stimulator and the arm 160.

The handheld controller/safety monitor 180 may include a switch that the patient can readily press to pause the treatment. Pressing the same switch twice in a row or for a certain duration can trigger the coil to retract to the resting position and stop the treatment. These controls are also accessible to the clinician in-person and remotely.

The system 100 may further include a mechanical arm release in which the shut-off switch releases the locking mechanism at the base rotational joint. The actuator may have a normally open operation, such that the lock will release with loss of power. The mechanical arm release may be driven by a digital signal from a microcontroller, which is also connected to ground with a pull-down resistor. The signal controls a transistor, such as a MOSFET, which activates the solenoid lock. When the mechanical arm release is released, it permits the arm 160 holding the TMS coil 140 to freely rotate away from the patient.

The system 100 may further include an at least 6-axis IMU (e.g., 3-axis accelerometer and 3-axis gyroscope) connected to a microcontroller contained within the handheld controller/safety monitor 180 to detect aberrant movement. The microcontroller continuously records acceleration and gyroscope values into a first-in, first-out (FIFO) buffer passed through a low-pass filter. A baseline buffer may be recorded just before the start of the treatment. During treatment, the real-time IMU buffer may be compared to the baseline buffer. Differences beyond a certain threshold during the treatment phase may signal an aberrant movement alarm. This may result in disarming and retracting of the TMS coil 140. The buffers are also processed through a Fast Fourier Transform to capture baseline tremor and possible seizure activity.

Machine learning models can also be used to detect and classify movement types (e.g., no movement, drop, normal/alert, possible seizure) and trigger associated alarms and, if necessary, contact the local or remote clinician.

Optionally, additional IMUs may be embedded into the armrests and seat cushion of the chair 120 to help detect patient movement. Based on the detected activity, an alarm for the specific condition may be triggered and treatment may be automatically paused or terminated.

Using the patient-facing camera on the graphical interface console 170, machine vision algorithms could be utilized to track head movement, gaze and iris positions, and recognize drowsiness, sleep, or abnormal head movements. Treatment may be paused or terminated, and alarms enabled, based on the event. Although it is not inherently dangerous for a patient to fall asleep, drowsy and sleep states are associated with reduced neuronal excitability, which might compromise efficacy of the treatment.

In the case where the handheld controller/safety monitor 180 is used, the system 100 may require the patient to be holding the handheld controller/safety monitor 180 for the stimulator to be enabled. If the treatment is in progress and the patient releases the handheld controller/safety monitor 180 beyond a certain period of time, as detected by a loss of contact with a capacitive touch sensor or release of force on the force sensing resistors on the surface of the controller, an alarm may be triggered and the treatment paused until the patient responds to prompts by the system 100 to resume the treatment, or else an emergency alarm may be triggered.

In some examples, the system 100 may include a pulse oximeter, heart rate sensor, temperature sensor, or any combination thereof, incorporated in the handheld controller/safety monitor 180 may trigger an alarm if the readings are outside of a certain threshold or deviate from the starting baseline values.

Using the combination of IMU data, vitals data, and head/iris/gaze tracking, one can create a more robust method of detecting the state of the patient and recognize potential alarm conditions.

In some examples, the TMS coil 140 may include a temperature sensor, which may be monitored to detect overheating, and can trigger pausing of treatment and activation of fans or other mechanisms to facilitate coil cooling. Treatment can resume when the coil temperature falls below a certain threshold.

If the navigation camera/machine vision of the machine vision navigation device 190 detects a certain fiducial marker (or other fiducial marker) within its field of view, an alarm condition or operational flag may be triggered. For example, there may be a fiducial marker printed on the surface of the seat, visible only when the patient is not in the chair. If such a marker is visible, it could trigger the arm 160 to stay in the resting/home position. There may be another fiducial marker incorporated into the headrest of the chair 120, such that when detected by the patient-facing graphical user interface, will indicate that the patient is not seated.

In some examples, a self-check using a built-in magnetic field sensor may ensure the TMS coil 140 is functioning within specifications. The sensor could be at a known, fixed location and rotation from the base of the TMS coil 140 holder (e.g., so in the case of the robotic arm, an automated self-test could be performed to ensure the TMS coil 140 output meets the intended specification). The TMS coil 140 may be disabled by the software if a deviation outside of a set tolerance is detected.

The system 100 may further include emergency alarms that may trigger the system 100 to prompt the user for a response on the graphical interface console 170, voice response, or other method of user interface. A response may be required of the user within a certain timeframe to resume the treatment, or otherwise trigger another alarm, like an emergency alert. For example, if seizure activity is suspected based on IMU data in combination with abnormal gaze or head movements (as detected by the machine vision module within the graphical interface) and the user does not provide a response within a certain period of time, an emergency phone call, text, and other alerts may be triggered. The GPS system and user information, such as address, can be automatically transmitted to emergency response.

Concerning trends on patient questionnaires may trigger associated alerts. For example, responses indicating potential acute suicidality may trigger an automated call to a crisis hotline or other emergency contact number (using built-in cellular module or user's cellular phone). Text or email alerts may also be sent to clinical providers and allow real-time remote monitoring and control by a clinician. Remote control may be enabled on-demand by the patient, triggered by an error/alarm condition, or required as prescribed by the clinician.

The system 100 may monitor treatment of a patient. Accurate positioning of the TMS coil 140 with respect to the target may be important for the quality of the stimulation, as small errors in coil placement can result in a significant reduction in cortical stimulation. The system 100 may incorporate a method to determine the current and overall “quality” of the TMS stimulation by considering the relative configuration of the TMS coil 140 with respect to the target. For instance, “100%” optimal coil alignment could be defined as when the center of the TMS coil 140 is directly over the target location, touching the target, tilted perpendicular to the target surface, and with the coil rotated 45 degrees from the mid-sagittal plane. Deviations from these factors may reduce the stimulation quality score. The score could be indicated qualitatively, such as “excellent,” “very good,” “good,” “fair,” “poor,” or quantitatively on a scale from 0-100. A score lower than a certain threshold over a certain period of time during the treatment could trigger an alarm and pausing the treatment. In the case of using a robotic arm, the error may be expected to be low since the arm would automatically compensate for head movements during the treatment.

Such a score could be calculated at a single point in time or as an overall measure using a moving average. An important variable may be the lack of contact between the target and the TMS coil 140, as the magnitude of the electric field generated by a TMS pulse decreases rapidly with distance from the center of the TMS coil 140 in the z-direction. Additionally, when the TMS coil 140 is off-center from the target location, the magnetic field may drop rapidly and can impact stimulation intensity. The tilt of the TMS coil 140 (e.g., tangential or not) and angle of rotation with respect to the mid-sagittal line may be other factors influencing stimulation quality (Janssen et al., 2015). The score can be stored for each treatment and overall for the entire course. This information may be helpful for the patient and the clinician.

Clinical outcomes may be tracked through self-report questionnaire responses and, optionally, clinician-administered scales. The system 100 may request user responses to questionnaires on a periodic basis to track clinical response. For example, in the case of treating someone for depression, the system 100 may ask the patient to complete a 9-item Patient Health Questionnaire (PHQ-9) or Beck Scale for Suicidal Ideation periodically. Cognitive screens and responses regarding PTSD symptoms, pain, and other clinically relevant data can also be displayed graphically on the graphical interface console 170 to clearly demonstrate trends.

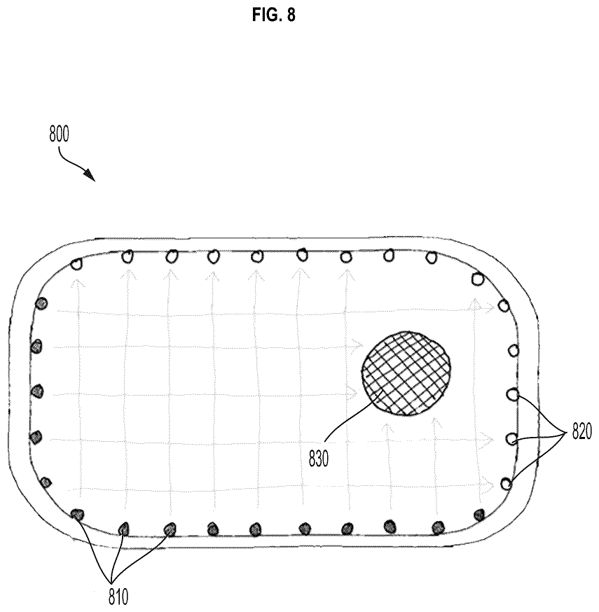

FIG. 8 depicts an example embodiment of a TMS coil 800 in accordance with embodiments described herein. The TMS coil 140 of FIG. 1 may implement the TMS coil 800 in some examples. In the TMS coil 800, an array of infrared emitters 810 and infrared receivers 820 can be placed along the outer bottom edge of the TMS coil 800. In one version, the emitters 810 are arranged opposite to corresponding infrared receivers 820. This type of arrangement may be used in some touchscreen displays. In the case there is an object touching the TMS coil 800, signals to 2 or more of the infrared receivers 820 will be at least partially obstructed. The pattern of obstruction can be used to deduce the location and size of contact with the coil 830. In another example, infrared emitters 810 and receivers 820 can be placed along the bottom of one edge of the TMS coil 800 in an alternating manner (e.g., emitter, receiver, emitter, receiver, etc.). When an object touches the surface, IR light is reflected from the object onto some of the *infrared receivers 820 in a diffuse pattern.

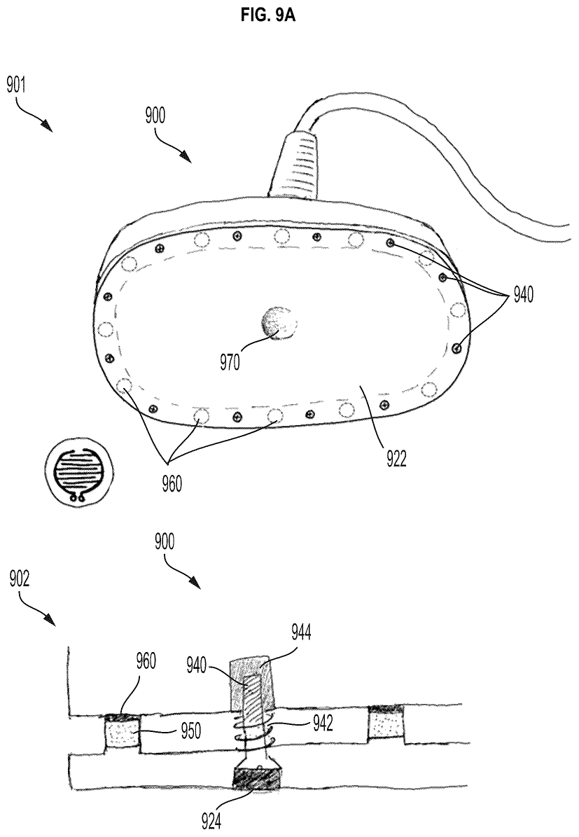

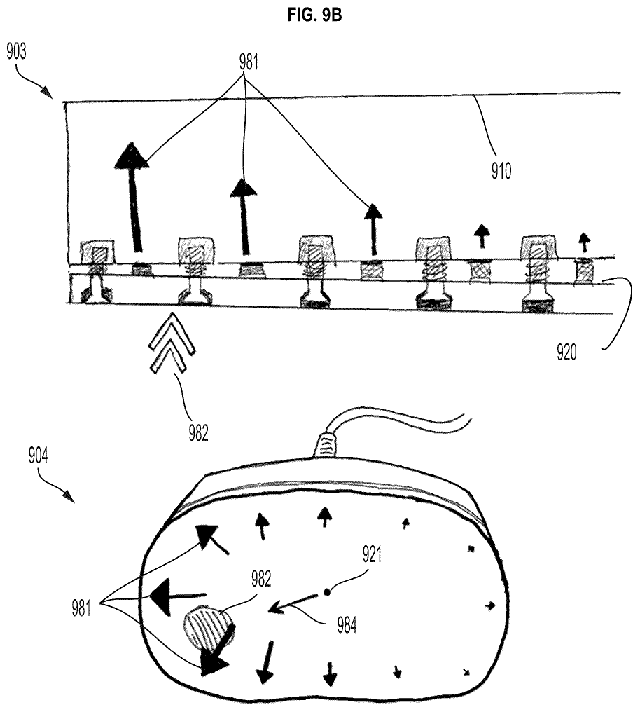

FIGS. 9A and 9B depict a various views 901, 902, 903, and 904 of an example embodiment of a TMS coil 900 in accordance with embodiments described herein. The TMS coil 140 of FIG. 1 may implement the TMS coil 900 in some examples. The TMS coil 900 may include an array of force sensing resistors along the outer bottom portion of the TMS coil 900 that is used to determine contact with the head and approximate location of contact and pressure applied.

The below figure shows a TMS coil in which the housing is divided into an upper 910 and lower portion 920. The functional part of the TMS coil 900 (loops of wire encased in resin or other substrate 922) is attached to the lower portion 920 of the housing. The lower portion 920 may be rigid and thin, so as to minimize distance from the wire loops 922 to the target surface. A ring of screws 940 secures the upper portion 910 to the lower portion 920. The upper portion 910 is securely attached to a support which holds the TMS coil 900 (e.g., an end effector of a robotic arm or a mechanical arm). The screws pass through springs 942 into screw inserts 944. The springs 942 may provide an opposing force and separates the upper portion 910 and bottom portion 920 of the housing by some small distance. There are recesses 924 in the lower portion 920 of the housing, so that when compressed, the screw head will not emerge beyond the housing. Additionally, along the same outer portion of the TMS coil housing between the upper portion 910 and the lower portion 920 are pieces of compressible foam or similar material 950 which may contact an array of force sensing resistors (FSRs) 960 attached to the upper portion 910 of the housing. One of the leads of each of the FSRs 960 is connected to a supply voltage. The other leads may each attached to input pins of an analog multiplexer with a pull-down resistor. The voltage at each of the input pins from the FSRs 960 can be used to approximate the pressure applied to each FSR 960 (e.g., higher voltage indicates a higher level of pressure).

In an example illustration, arrows 981 depict the relative value of the analog readings from each of the FSRs 960 when force is applied to the lower portion 920 of the housing at location 982. Reading from each FSR 960 can be converted into a vector with the direction portion of the vector being the relative location from the center 921 of the lower portion 920 of the coil housing. A vector sum 984 for all the FSR readings can be calculated by a microcontroller. The vector sum 984 can thus permit determination of approximate location of contact. The average FSR reading can be used to approximate degree of pressure applied.

The bottom portion of the housing may include a small indentation 970 which can be used to calibrate the FSR readings. For example, with the coil facing down and not touching anything, the FSRs 960 will each pass some small voltage due to pressure from the compressible material opposing it. These values can be mapped to a “zero” level for each FSR 960. When an object, shaped to couple with the small indentation 970 is pressed with moderate force against the small indentation 970 in an orthogonal direction, the FSRs will read a higher voltage, which can be mapped to a “max” level for each FSR 960. This process helps to establish reference values that can be used for subsequent determination of coil contact location and relative pressure.

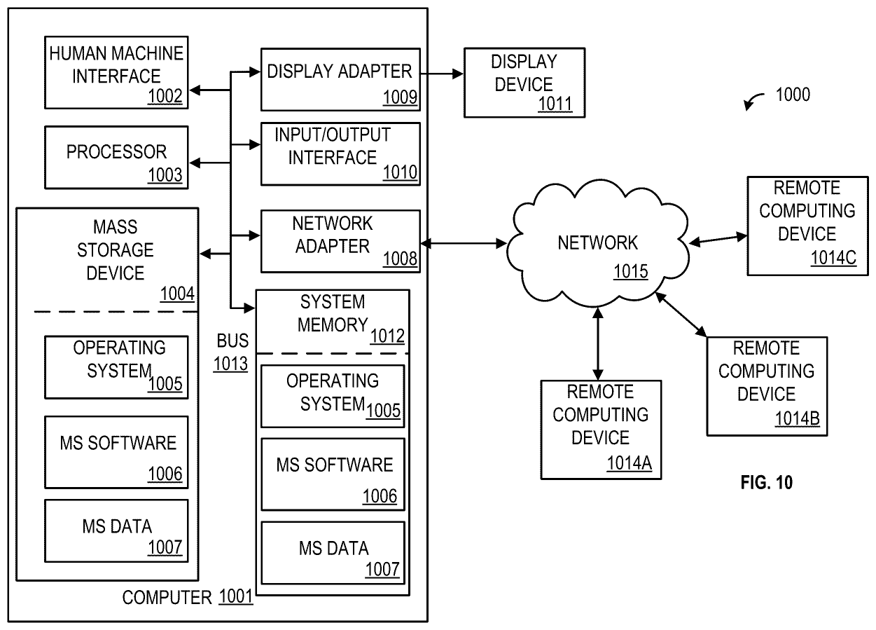

FIG. 10 depicts a system 1000 including a computing device 1001 for use with the system 100 of FIG. 1 in accordance with embodiments described herein. The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as MS data 1007 and/or program modules such as operating system 1005 and MS software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and MS software 1006 may be stored on the mass storage device 1004. One or more of the operating system 1005 and MS software 1006 (or some combination thereof) may comprise program modules and the MS software 1006. MS data 1007 may also be stored on the mass storage device 1004. MS data 1007 may be stored in any of one or more databases known in the art. The databases may be centralized or distributed across multiple locations within the network 1015.

A user (e.g., the clinician) may enter commands and information into the computing device 1001 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 via a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 via an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 via Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014A,B,C. A remote computing device 1014A,B,C may be a personal computer, computing station (e.g., workstation), portable computer (e.g., laptop, mobile phone, tablet device), microcontroller, smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014A,B,C may be made via a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of MS software 1006 may be stored on or sent across some form of computer readable media. Any of the disclosed methods may be performed by processor-executable instructions embodied on computer readable media.

In some embodiments, the computing device 1001 may be electronically connected to one or more imaging devices, for example a device or system for performing one or more of computed tomography, radiography, medical resonance imaging, or ultrasound.

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

### Examples

Example 1 is a system, comprising: a magnetic stimulation coil; and a machine vision navigation device comprising a camera configured to capture successive still images and/or to stream video indicating position of the magnetic stimulation coil, wherein the machine vision navigation device is configured to determine, based on the successive still images and/or the video, the position of the magnetic stimulation coil relative to a target treatment area of a patient, and cause, based on the position of the magnetic stimulation coil relative to the target treatment area of the patient, display of an indication of the position of the magnetic stimulation coil relative to the target treatment area of the patient.

In Example 2, the subject matter of Example 1 includes, wherein the machine vision navigation device is further configured to identify, based on detection of a fiducial marker in the successive still images and/or the video, the target treatment area of the patient.

In Example 3, the subject matter of Example 2 includes, wherein the machine vision navigation device is further configured to determine, based on a location of the fiducial marker within the successive still images and/or the video, a distance between the target treatment area and the magnetic stimulation coil.

In Example 4, the subject matter of Examples 2-3 includes, wherein the machine vision navigation device is further configured to determine, based on an orientation of the fiducial marker within the successive still images and/or the video, a an orientation difference between the target treatment area and the magnetic stimulation coil.

In Example 5, the subject matter of Examples 1˜4 includes, wherein the machine vision navigation device is further configured to cause, based on a determination that the position of the magnetic stimulation coil relative to the target treatment area of the patient satisfies a threshold, repetitive transcranial magnetic stimulation at the target treatment area of the patient using the magnetic stimulation coil.

In Example 6, the subject matter of Examples 1-5 includes, wherein the machine vision navigation device is further configured to cause, based on a determination that the position of the magnetic stimulation coil relative to the target treatment area of the patient does not satisfy a threshold, the magnetic stimulation coil to move toward the target treatment area of the patient.

In Example 7, the subject matter of Examples 1-6 includes, a robotic arm, wherein the magnetic stimulation coil is mounted to the robotic arm, wherein the machine vision navigation device is further configured to cause, based on a determination that the position of the magnetic stimulation coil relative to the target treatment area of the patient does not satisfy a threshold, the robotic arm to articulate to direct the magnetic stimulation coil toward the target treatment area of the patient.

In Example 8, the subject matter of Examples 1-7 includes, a movable arm, wherein the magnetic stimulation coil is mounted to the movable arm, wherein the machine vision navigation device is further configured to cause, based on a determination that the position of the magnetic stimulation coil relative to the target treatment area of the patient does not satisfy a threshold, display of instructions to articulate the movable arm to direct the magnetic stimulation coil toward the target treatment area of the patient.

In Example 9, the subject matter of Examples 1-8 includes, a handheld controller comprising a switch, wherein the machine vision navigation device is further configured to cause, based on receipt of an indication of activation of the switch, transcranial magnetic stimulation at the target treatment area of the patient to stop.

In Example 10, the subject matter of Example 9 includes, wherein the machine vision navigation device is further configured to cause, based on receipt of an indication of a second activation of the switch, transcranial magnetic stimulation at the target treatment area of the patient to restart.

Example 11 is an apparatus, comprising: one or more processors; a magnetic stimulation coil; a camera configured to capture successive still images and/or to stream video indicating position of the magnetic stimulation coil; memory storing processor executable instructions that, when executed by the one or more processors, cause the apparatus to: determine, based on the successive still images and/or the video, the position of the magnetic stimulation coil relative to a target treatment area of a patient; and cause, based on the position of the magnetic stimulation coil relative to the target treatment area of the patient, display of an indication of the position of the magnetic stimulation coil relative to the target treatment area of the patient.

In Example 12, the subject matter of Example 11 includes, wherein the processor executable instructions further cause the apparatus to identify, based on detection of a fiducial marker in the successive still images and/or the video, the target treatment area of the patient.

In Example 13, the subject matter of Example 12 includes, wherein the processor executable instructions further cause the apparatus to determine, based on a location of the fiducial marker within the successive still images and/or the video, a distance between the target treatment area and the magnetic stimulation coil.

In Example 14, the subject matter of Examples 12-13 includes, wherein the processor executable instructions further cause the apparatus to determine, based on an orientation of the fiducial marker within the successive still images and/or the video, a an orientation difference between the target treatment area and the magnetic stimulation coil.

In Example 15, the subject matter of Examples 11-14 includes, wherein the processor executable instructions further cause the apparatus to cause, based on a determination that the position of the magnetic stimulation coil relative to the target treatment area of the patient satisfies a threshold, repetitive transcranial magnetic stimulation at the target treatment area of the patient using the magnetic stimulation coil.

In Example 16, the subject matter of Examples 11-15 includes, wherein the processor executable instructions further cause the apparatus to cause, based on a determination that the position of the magnetic stimulation coil relative to the target treatment area of the patient does not satisfy a threshold, the magnetic stimulation coil to move toward the target treatment area of the patient.

In Example 17, the subject matter of Examples 11-16 includes, wherein the processor executable instructions further cause the apparatus to cause, based on a determination that the position of the magnetic stimulation coil relative to the target treatment area of the patient does not satisfy a threshold, a robotic arm to which the magnetic stimulation coil is affixed to articulate to direct the magnetic stimulation coil toward the target treatment area of the patient.

In Example 18, the subject matter of Examples 11-17 includes, wherein the processor executable instructions further cause the apparatus to cause, based on a determination that the position of the magnetic stimulation coil relative to the target treatment area of the patient does not satisfy a threshold, display of instructions to articulate a movable arm to which the magnetic stimulation coil is affixed to direct the magnetic stimulation coil toward the target treatment area of the patient.

In Example 19, the subject matter of Examples 11-18 includes, wherein the processor executable instructions further cause the apparatus to cause, based on receipt of an indication of activation of a switch of a handheld controller, transcranial magnetic stimulation at the target treatment area of the patient to stop.

In Example 20, the subject matter of Example 19 includes, wherein the processor executable instructions further cause the apparatus to cause, based on receipt of an indication of a second activation of the switch, transcranial magnetic stimulation at the target treatment area of the patient to restart.

Example 21 is a method, comprising: receiving successive still images and/or a video stream indicating a position of a magnetic stimulation coil; determining, based on the successive still images and/or the video stream, the position of the magnetic stimulation coil relative to a target treatment area of a patient; and causing, based on the position of the magnetic stimulation coil relative to the target treatment area of the patient, display of an indication of the position of the magnetic stimulation coil relative to the target treatment area of the patient.

In Example 22, the subject matter of Example 21 includes, determining, based on detection of a fiducial marker in the successive still images and/or the video stream, the target treatment area of the patient.

In Example 23, the subject matter of Example 22 includes, determining, based on a location of the fiducial marker within the successive still images and/or the video stream, a distance between the target treatment area and the magnetic stimulation coil.

In Example 24, the subject matter of Examples 22-23 includes, determining, based on an orientation of the fiducial marker within the successive still images and/or the video stream, an orientation difference between the target treatment area and the magnetic stimulation coil.

In Example 25, the subject matter of Examples 21-24 includes, causing, based on a determination that the position of the magnetic stimulation coil relative to the target treatment area of the patient satisfies a threshold, repetitive transcranial magnetic stimulation at the target treatment area of the patient using the magnetic stimulation coil.

In Example 26, the subject matter of Examples 21-25 includes, causing, based on a determination that the position of the magnetic stimulation coil relative to the target treatment area of the patient does not satisfy a threshold, the magnetic stimulation coil to move toward the target treatment area of the patient.

In Example 27, the subject matter of Examples 21-26 includes, causing, based on a determination that the position of the magnetic stimulation coil relative to the target treatment area of the patient does not satisfy a threshold, a robotic arm to which the magnetic stimulation coil is affixed to articulate to direct the magnetic stimulation coil toward the target treatment area of the patient.

In Example 28, the subject matter of Examples 21-27 includes, causing, based on a determination that the position of the magnetic stimulation coil relative to the target treatment area of the patient does not satisfy a threshold, display of instructions to articulate a movable arm to which the magnetic stimulation coil is affixed to direct the magnetic stimulation coil toward the target treatment area of the patient.

In Example 29, the subject matter of Examples 21-28 includes, causing, based on receipt of an indication of activation of a switch of a handheld controller, transcranial magnetic stimulation at the target treatment area of the patient to stop.

In Example 30, the subject matter of Example 29 includes, causing, based on receipt of an indication of a second activation of the switch, transcranial magnetic stimulation at the target treatment area of the patient to restart.

Although the foregoing invention has been described in some detail by way of illustration and example for purposes of clarity of understanding, certain changes and modifications may be practiced within the scope of the appended claims.

What is claimed is:

1. A system, comprising:
a magnetic stimulation coil; and
a machine vision navigation device comprising a camera configured to capture successive still images and/or to stream video indicating position of the magnetic stimulation coil, wherein the machine vision navigation device is configured to determine, based on the successive still images and/or the video, the position of the magnetic stimulation coil relative to a target treatment area of a patient, and cause, based on the position of the magnetic stimulation coil relative to the target treatment area of the patient, display of an indication of the position of the magnetic stimulation coil relative to the target treatment area of the patient.

2. The system of claim 1, wherein the machine vision navigation device is further configured to identify, based on detection of a fiducial marker in the successive still images and/or the video, the target treatment area of the patient.

3. The system of claim 2, wherein the machine vision navigation device is further configured to determine, based on a location of the fiducial marker within the successive still images and/or the video, a distance between the target treatment area and the magnetic stimulation coil.

4. The system of claim 2, wherein the machine vision navigation device is further configured to determine, based on an orientation of the fiducial marker within the successive still images and/or the video, an orientation difference between the target treatment area and the magnetic stimulation coil.

5. The system of claim 1, wherein the machine vision navigation device is further configured to cause, based on a determination that the position of the magnetic stimulation coil relative to the target treatment area of the patient satisfies a threshold, repetitive transcranial magnetic stimulation at the target treatment area of the patient using the magnetic stimulation coil.

6. The system of claim 1, wherein the machine vision navigation device is further configured to cause, based on a determination that the position of the magnetic stimulation coil relative to the target treatment area of the patient does not satisfy a threshold, the magnetic stimulation coil to move toward the target treatment area of the patient.

7. The system of claim 1, further comprising a robotic arm, wherein the magnetic stimulation coil is mounted to the robotic arm, wherein the machine vision navigation device is further configured to cause, based on a determination that the position of the magnetic stimulation coil relative to the target treatment area of the patient does not satisfy a threshold, the robotic arm to articulate to direct the magnetic stimulation coil toward the target treatment area of the patient.

8. The system of claim 1, further comprising a movable arm, wherein the magnetic stimulation coil is mounted to the movable arm, wherein the machine vision navigation device is further configured to cause, based on a determination that the position of the magnetic stimulation coil relative to the target treatment area of the patient does not satisfy a threshold, display of instructions to articulate the movable arm to direct the magnetic stimulation coil toward the target treatment area of the patient.

9. The system of claim 1, further comprising a handheld controller comprising a switch, wherein the machine vision navigation device is further configured to cause, based on receipt of an indication of activation of the switch, transcranial magnetic stimulation at the target treatment area of the patient to stop.

10. The system of claim 9, wherein the machine vision navigation device is further configured to cause, based on receipt of an indication of a second activation of the switch, transcranial magnetic stimulation at the target treatment area of the patient to restart.

11. An apparatus, comprising:
one or more processors;
a magnetic stimulation coil;
a camera configured to capture successive still images and/or to stream video indicating position of the magnetic stimulation coil;
memory storing processor executable instructions that, when executed by the one or more processors, cause the apparatus to:
determine, based on the successive still images and/or the video, the position of the magnetic stimulation coil relative to a target treatment area of a patient; and
cause, based on the position of the magnetic stimulation coil relative to the target treatment area of the patient, display of an indication of the position of the magnetic stimulation coil relative to the target treatment area of the patient.

12. The apparatus of claim 11, wherein the processor executable instructions further cause the apparatus to identify, based on detection of a fiducial marker in the successive still images and/or the video, the target treatment area of the patient.

13. The apparatus of claim 12, wherein the processor executable instructions further cause the apparatus to determine, based on a location of the fiducial marker within the successive still images and/or the video, a distance between the target treatment area and the magnetic stimulation coil.

14. The apparatus of claim 12, wherein the processor executable instructions further cause the apparatus to determine, based on an orientation of the fiducial marker within the successive still images and/or the video, an orientation difference between the target treatment area and the magnetic stimulation coil.

15. The apparatus of claim 11, wherein the processor executable instructions further cause the apparatus to cause, based on a determination that the position of the magnetic stimulation coil relative to the target treatment area of the patient satisfies a threshold, repetitive transcranial magnetic stimulation at the target treatment area of the patient using the magnetic stimulation coil.

16. A method, comprising:
receiving successive still images and/or a video stream indicating a position of a magnetic stimulation coil;
determining, based on the successive still images and/or the video stream, the position of the magnetic stimulation coil relative to a target treatment area of a patient; and
causing, based on the position of the magnetic stimulation coil relative to the target treatment area of the patient, display of an indication of the position of the magnetic stimulation coil relative to the target treatment area of the patient.

17. The method of claim 16, further comprising determining, based on detection of a fiducial marker in the successive still images and/or the video stream, the target treatment area of the patient.

18. The method of claim 17, further comprising determining, based on a location of the fiducial marker within the successive still images and/or the video stream, a distance between the target treatment area and the magnetic stimulation coil.

19. The method of claim 17, further comprising determining, based on an orientation of the fiducial marker within the successive still images and/or the video stream, an orientation difference between the target treatment area and the magnetic stimulation coil.

20. The method of claim 16, further comprising causing, based on a determination that the position of the magnetic stimulation coil relative to the target treatment area of the patient satisfies a threshold, repetitive transcranial magnetic stimulation at the target treatment area of the patient using the magnetic stimulation coil.
